(19) United States
(12) Patent Application Publication
(10) Pub. No.: US 2025/0161103 A1
(43) Pub. Date: May 22, 2025

(54) DNA Fixing Machine

(71) Applicant: Francisco Collado, Murrieta, CA (US)

(72) Inventor: Francisco Collado, Murrieta, CA (US)

(21) Appl. No.: 18/808,035

(22) Filed: Aug. 18, 2024

(51) Int. Cl. A61F 7/00

(52) U.S. Cl. CPC A61F 7/00

(57) ABSTRACT

The DNA fixing machine will be used to activate the immune system to fix all types of diseases (cancer, diabetics, etc.). All diseases past, present and future are caused by a mutation in the DNA. When the immune system cannot fix this mutation, the results of this is called a disease. A difference of all the medical treatments now that provides the chemical or biological agents missing, the DNA Fixing Machine will be fixed permanently in minutes and without side effects. The DNA machine will not only be able to fix the cell's DNA mutations that caused diseases, but it can also be used to activate the STEM cells and replace any missing limb or cells that don't reproduce under normal conditions (paralysis, brain problems, etc.). It can also be used to clean problems in the blood by changing the velocity of the blood from laminar to turbulent.

Cells are divided into two main classes, whether they contain a nucleus. Prokaryotic cells (bacteria) lack a nucleus envelope; eukaryotic cells have a nucleus in which the genetic material is separated from the cytoplasm. The DNA fixing machine is for certain Eukaryotic cells (humans and animals).

There are four types of Eukaryotic cells: Animals, Fungi, Plants and Protists. Their genetic material is organized into chromosomes (found in the nucleus and they carried the genetic information). Diseases in Fungi and Protists are not important to us and Plants because of the lack of mobility and because they are missing important organs (like heart, lungs, etc.), their diseases are not a big problem. The plant world is not important to us.

The DNA fixing machine can be used to fix medical problems caused by mutations inside the body. The DNA fixing machine is the equivalent to a heat pump in the HVAC world, where the same machine can cool or heat, depending on the need. It is designed to work inside the body; pass the three layers of the skin.

The best defense against diseases is the immune system. The immune system is a very complicated system consisting of different layers of protection, but it is not a perfect one. In a healthy person an average of 35,000 DNA mistakes are corrected per day. When the immune system fails to correct mistakes, we have a problem and we call this problem a disease. Depending on the level and location of DNA mutation, the disease will produce different effects on the person or animal. We gave these symptoms a name; it could be cancer, diabetics, blindness, mental problems, etc. They all have the root (DNA mutations), so they can be fixed by the same treatment.

For the purpose of the DNA Machine, diseases can be divided into 3 different types, but before getting into this, let's see what a disease is.

The DNA in humans (the same idea is applicable to the rest of the animal kingdom) consists of four bases called G, A, C and T and two double stranded helix held together by weak hydrogen bonds. Under normal or healthy conditions the T is paired with A and the G is paired with the C.

Each set of three bases in a sequence along one strand of DNA spells a specific genetic word, called a Codon. When there are enough words in a row to have biological meaning, we call that a gene. Each piece of human DNA is long enough to spell thousands of genes.

### First Disease Type (Mutation at Cell Division):

The cell division (human, similar concept for other animals) consists of a few phases. The first phase is the G1 phase, which is the phase where cell growth and it prepares for DNA replication. There is a checkpoint at the beginning of this phase called the G1 arrest, where the cell checks for DNA damage and if one is found; the cell undergoes repair mechanisms or follows the apoptotic pathway (the cell gets killed). The immune system has the capability to replace cells that were killed. It is important to note here that if the cell cannot be repaired; the immune system is killed and the cell is replaced. At the end of the G1 phase there is another check point called the G1 restriction point, where the cell checks for DNA damage again and if one is found; it stops the cell division and it moves to the GO phase (where cells are in the non-division state). Important here to note that a damaged cell can return to its original state; it is a reversible process. This is done by returning the cell to its original temperature.

After the G1 phase, the cell moves into the S phase, where DNA replication occurs.

In the S phase the cell synthesizes a complete copy of the DNA in its nucleus. At the end of the S phase there is another checkpoint called S arrest. Here the cell checks for DNA damage again and if one is found; it stops cell duplication until the DNA damage is fixed. From the S phase it moves into the G2 phase. Cells can be fixed, even the DNA is damaged.

In the G2 phase the cell prepares for mitosis (cell duplication). At the beginning of the G2 phase there is another checkpoint called G2 arrest, where the cell checks for DNA damage and if one is found, it stops cell duplication until the mistake is fixed.

After the G2 phase the cell goes into the M phase where mitosis occurs, followed by cytokinesis (cytoplasmic division).

For the major part of G2 phase, all the way to the cytokinesis phase, there is no checkpoints, so if the cell is hit with short wavelengths (X-rays, gamma-ray radiation, ultraviolet rays, etc.), chemicals or biological agents mutations to the DNA will occur; this mutation will be passed to the new daughter cell and it will accepted by the immune system as a “normal” or healthy cell, even if it is not. If the mutation occurs in part of the cell that is active, it could affect the behavior of the cell and a disease could develop.

### Second Disease Type (Mutation at Non-Dividing Cells)

There are cells that are dormant on quiescent states (called G0); they don't divide. A good example of this is neuron cells (nerve cells that send messages all over your body to allow you to do everything from breathing to talking, eating, walking, and thinking, etc.). Centrosomes are small organelles present beside the nucleus of the cell; the centrioles (barrel-shaped organelles) in the centrosome initiate spindle formation for cell division to take place. Neurons lack this organelle and hence, lack centrioles. This is the reason they and other cells are on the G0 state all the time and cannot divide. The STEM cell system is able to reproduce missing dormant cells if the correct stimulus is provided; one important function of the cell is that each cell knows its place on the body.

The immune system has a system in place to protect cells in the G0 phase from DNA mutations. When a mutation of the DNA of a cell in the G0 phase occurs; MHC-1 molecules are expressed on the cell surface to signal the natural killer cells and the T-cells (both white blood cells of the immune system) that something is wrong with the cell and it needs to be killed and replaced.

MHC-1 molecule is a chain of 350 amino acids, and it is very temperature dependent. Under “normal cell temperatures” MHC-1 is continuously delivered to the cell surface to signal the immune system if there is a problem. At higher temperatures MHC-1 is stopped and a lower temperature MHC-1 is expanded beyond their lengths to work as designed resulting in a useless MHC-1.

In the mechanical plumbing and electrical world, if something works between the plus or minus 10% of the design number, the system will work as designed. In the microscopic field of the cells; the cell's volume will work as designed as long as the original volume of the cell is between plus or minus 10%. A good example of this is diabetics type 2, in persons with type 2 diabetics the reduction in Beta cells (cells that make insulin, a hormone that controls the level of glucose (a type of sugar)) in the blood was reduced by 37% if the volume of the original Beta cells was bigger than 10%.

Mutations on dormant cells that change the volume of the cells by plus or minus 10% will cause a malfunction on the MHC-1 molecule causing a malfunction of the immune system where cells are killed and not replaced. This is the second type of disease.

### Third Disease Type (Hereditary Mutations)

Gametes are an organism's reproductive cells. In human's fertilization, an egg cell and a sperm cell combine to make one cell that will divide and differentiate to create a human body.

We are made of a single cell that is divided and multiplied. All the cells are basically the same, but the majority of the bases (letters) on the DNA are turned off; this is what makes a foot cell different from an eye cell for example. This is the reason why a mutation that occurs in a “dormant or turn off” part of the DNA is not a problem.

The gametes have a program that tells each cell how to divide and what bases to turn on and which ones need to turn off; this is given to the DNA of each cell.

The gametes can be altered by radiation, chemicals and some biological agents. In the case of hereditary diseases they will alter the computer program of the gamete's DNA to create a mutation after a certain number of cell divisions. This mutation will affect the function of the cells or organ creating a disease but it will be accepted by the immune system as a healthy cell or organ because it was in the original computer program. A good example of this is “cancer in families”; for example a mother has breast cancer at 25 years old, and her daughter will have breast cancer at around 25 years old. This is the third type of disease.

Let's look at what a DNA mutation is.

The DNA is the computer program of the cell. In the real world you always want to keep the original computer program safe; cells do the same. To keep the original program safe, cells provide messages to the organelles of the cells by using the messenger RNA or mRNA. The cell is a closed system, so for the cell to function correctly the messenger needs to be destroyed once it is delivered, otherwise there will be multiple messengers being delivered to the same organelle at the same time with different instructions. This will destroy the cell. The mRNA has four bases (same as the DNA) but here we have A, G, C and U (it replaces T).

There are different kinds of mutations, one could be eliminating a base or letter from the DNA and another will be replacing a letter with another one and depending on the timing the letter U of the mRNA can be inserted into the DNA of the cell. Different energy is needed for different mutations; this breaking and creating of hydrogen bonds results in a change of cell temperature compared to the original cell temperature.

If the mutation is in a base that is turning off, it is not an issue. Replacing a lamp in a turn off light fixture won't change the lumens of the room, but if the mutation is on an active base, it will change the genetic code writing and this could change the instruction to a certain organelle and it could be a problem, and a problem is what we call a disease.

Cells are a closed system and they work by the principle of osmotic pressure. There are hundreds of ions pump, pumping elements into the cell and hundreds of ions pumps pumping elements out of the cell. All these pumps have variable frequency drives, so if one pump slows down another will need to speed up to keep the pressure constant, otherwise the cell will died (this is the principle of osmotic pressure, it needs to be constant all the time). One of the ways the immune system fixes a DNA mutation that cannot be fixed is by changing the osmotic pressure of the cell, so it dies (good examples of this are viruses). Remember the death cell will be replaced with one new original one (the power of STEM cells).

This means that changes on cells are isobaric (constant pressure) and by the first law of thermodynamics, a slow isobaric process is reversible.

The majority of the diseases are slow isobaric processes (for example cancer needs at least 6 different cell mutations to change from a “normal” cell to a cancer cell), so they are reversible. A reversible process means that a cell mutation if it is returned to its original volume or temperature, the problem is fixed.

DNA fixing machine uses:

The first use of the DNA fixing machine is to return disease or sick cells to its original state to reverse the problem.

Unfortunately, not all the diseases are reversible, as in every field they are exceptions. The DNA machine can be used here to kill the disease cells by increasing or decreasing the cell temperature for a certain amount of time; this will be the second application of the DNA fixing Machine.

Cells follow the ideal equation PV=NRT; where P is the osmotic pressure, N (number of moles) and R (is the universal constant); all these three variables are constant values, so they can be eliminated from the equation and we get:
V=T (volume is a function of temperature, or temperature is a function of volume).

Volume is very important to the cell. Each cell has a limit of surface area to volume ratio to ensure that the exchange of resources and waste occurs quickly enough for the cell to survive and function correctly. Most of the human cells are spherical shape; the surface area formula for a sphere is 4*PI*R^2 and the formula for its volume is 4*PI*R^3/3. If we divide the surface area by the volume, we get 3/R as the volume of the cell increases, the surface area to volume gets smaller and this means there is less surface area for materials to be transported as efficiently as possible in and out of the cell; making the cell less efficient. Cells can increase surface area by folding the plasma membrane to compensate for the increase in volume size and still be efficient. As the volume of the cell keeps increasing; it will be insufficient surface area to support the cell's increased volume and the cell will either divide or die.

Reducing the cell volume will make the cell more efficient at the beginning because they will have more surface area for the exchange of chemicals, but as the volume is reduced further; it will increase the loss of water and dissolved substances causing temperature cell problems. Increasing the volume too much or reducing the volume too much is a problem.

The bases of the DNA are being kept together by hydrogen bonds. The breaking of hydrogen bonds needs energy (called endothermic reaction) and energy released during formation of hydrogen bonds (called exothermic). For a DNA mutation to occur, breaking of hydrogen bonds or breaking and formation of hydrogen bonds need to occur. These breakings and bonding formations change the original temperature of the cell, and this means the original volume of the cell changed too.

Sick cells have bigger or smaller volume than their original healthy cell. Also, by the formula above; a sick cell will have a smaller or higher temperature than a normal cell.

It is very difficult to find a change of volume in cells if you don't know where to look, especially if we consider that there are a million cells in a pin head. It is easy to find a group of cells that have higher temperature than the surrounding ones (or the original ones in the case of metastatic cancer). A higher or lower cell temperature means there is a problem or a disease. For a problem or disease to occur, a lot (probably millions) of the same type of cells need to be mutated (the exception is cancer where one mutated cell keeps dividing non stop causing problems). Temperature difference of the cells can be detected with the technology we have available now (medical grade thermocouples for example). As we said before, this is the first application of the DNA fixing machine, returning the cell to its original volume and temperature, so the immune system can fix the problem.

Bear in mind what the DNA fixing machine is trying to do (first application); it has occurred naturally. A person was hit by electricity and its cancer tumor disappeared, a girl fell from a tree and her sick and incurable stomach cells were fixed; just to mention a couple of examples. We called these cases miracles; but the miracle here was that the external factors changed the blood flow amount (increased or decreased depending on the problem) to the “sick” cells and they got the right temperature for a few seconds or minutes and this was enough to fix the problem. One of the important abilities of the immune system is that it has the ability that once it detects that one sick cell in a group of cells is fixed; it knows that all the other sick cells need to be fixed; there is no better medicine than your own immune system.

The DNA fixing machine can also be used to kill cells that cannot be fixed by returning the sick cells to its original temperature (non reversible problems). Healing the sick cells temperature can be kept at higher or lower temperatures for a certain amount of time to kill the cells and the immune system will replace them with healthy cells. This will be the second application of the DNA Fixing Machine.

The DNA fixing machine can also be used to start cell duplication or activate STEM cells. STEM cells are the body's raw materials, cells from which all other cells with specialized functions are generated. A difference of a specialized cell that can only divide 26 times, STEM cells can divide forever; one cell will be the specialized cell and the other one will remain as a STEM cell. STEM cells are found all over the body.

This will be the third application of the DNA Machine, it can be used to duplicate cells or activate the STEM cell process to provide the body with the cell or organs it needs or are missing. Cells have memory so they know where they are and how many they need to work as designed. Good examples of this application will be missing limbs, Alzheimer's disease, paralysis, etc.

For a machine to work as designed, it needs good maintenance. You don't go and overhaul the whole machine; you maintain its critical parts. In moving, living organisms the part that needs maintenance is the circulatory system.

The circulatory system carries oxygen, nutrients, and hormones to cells, and removes waste products. There are four basic problems caused by the circulatory system malfunction. The first is Aneurysms; it occurs when an artery wall weakens and enlarges. The weak spot may tear, causing a life-threatening rupture. Fats build up on the inside of artery walls resulting in damage and weakening of blood vessels is the main cause for this. The other circulatory system disease is high blood pressure; the force of the blood pushing against the artery walls is consistently too high. Again plaque buildup in the arteries is the main cause of high blood pressure, this plaque buildup restricts the flow of blood, so to compensate for this the heart pumps harder. The third circulatory system problem is plaque deposits in the arterial walls resulting in high cholesterol levels. Chemical signals that are generated as a result of injury cause white cells to attach to the arterial wall, where they collect cholesterol and other fatty material, eventually forming plaque and making the heart work harder. The last circulatory system problem is venous disease. Venous diseases tend to affect veins in the lower body and occur when blood can't flow back to the heart and pools in leg veins. Vein valves ensure that blood flows in one direction toward your heart, if the vein valves are damaged (partly closed), they won't allow enough blood to return causing this disease.

The best way to remove “waste” at pipe walls and open closed valves is to increase the water velocity, so I will use the same cleaning process here. To remove plaque deposits at the arteries wall and to open partially closed vein valves, we will increase the velocity of the blood (from laminar to turbulent) and the DNA fixing machine can do this. This will fix the circulatory problems 1, 2, 3 and 4 above. This is the fourth application of the DNA fixing machine.

There are special cases where the disease is not caused by a DNA mutation but by changes in cell temperature where the volume is between the 10% margin of error, so the immune system assumes everything is fine. A good example of this are sleeping viruses that get inside the cell increasing its temperature but not big enough to activate the immune system. The DNA fixing machine can fix these problems, once the cell or cells with higher temperature are identified.

There are many different variables that need to be calculated at the same time for the DNA fixing machine to work. For example in cancer all the functions are the same, but the size of the tumor and the type of the tumor (I, II, III or IV) is different and this will affect the calculations. A computer program was developed that will ask standard medical questions that takes into account all these variables and tell you how to set up the machine and for how long it needs to operate to fix the problem. Cancer changes from person to person and no two cancers are the same, even if they are the same type and in the same location. The DNA fixing machine will take this into account.

For other diseases, it makes a big difference if you are skinny/muscular or an obese person. A new different computer program will be required; so are the cases for the other applications of the DNA fixing machine.

Due to the big number of variables, there is not a magic ballet, so to be able to use the same machine, it needs to adapt from person to person and from disease to disease. The DNA fixing Machine will be a flexible machine (second part). The last part of the machine (the wire that will go inside the body); will be the removable part of the machine and it can be cleaned using an Autoclave machine (clean steam), so it can be used over and over again.

The first part will be the storage coil; it will provide the required temperature the cell needs to fix the problem and it is designed to keep the needed temperature constant

The DNA fixing machine is a medical device (type II), and it needs to be used inside a medical facility by professional medical people.

It needs to operate inside a constant room temperature. It is recommended to keep the room temperature at 22.5° C. because this is the ideal room temperature for the immune system to work at optimum conditions (the whole idea of the DNA fixing machine is to fix the medical issues by using the immune system). Of course, 22.5° C. is not required; you can input a different room temperature in the computer program and it will calculate what to do using the input room temperature number given.

This room temperature needs to be independent of the whole facility; it needs its own thermostat and HVAC equipment. Thermoelectric systems work independent of other HVAC systems, so there is no problem having two HVAC systems in the same room; they won't affect each other.

It is recommended that medical design facilities with the additional equipment required be designed by a professional engineer in the HVAC design consultant field.

1. The DNA fixing machine can change the “sick” cells to its original healthy cells (original temperature), and after a few minutes fix the problem. All this is done by conduction. This is the first use of the DNA fixing machine.
Unfortunately, there are diseases that for some reason are not reversible. In these cases the “sick” cells can be killed by necrosis or by apoptosis. Necrosis occurs when the cell temperature is kept at minus 50 degree Celsius for a certain amount of time (ice crystals form on the membrane of the cells, damaging and killing the cell), or when the temperature is kept at 50 degrees Celsius for certain amount of time (too little blood flow, killing the cells). The computer program will calculate the amount of time needed (it depends on the type and size of the sick cells) to kill the damaged cells by conduction. Apoptosis occurs when the cells are kept at 43 degrees Celsius for a certain amount of time or when the cells are kept at 6 degrees Celsius for a certain amount of time. This is what your immune system does when the cells are infected with viruses or when they are not functioning as designed. The DNA fixing machine will keep the “sick” cells at the required temperature for Apoptosis by using conduction. This will be the second use of the DNA fixing machine.
The third use of the DNA fixing machine is to activate the STEM Cells. STEM cells are activated naturally by the body by two mechanisms. One is tissue disruption and the other is upon injury. Stem cells can also be activated by “heat shock”, which is keeping the cells at 39-42 degrees Celsius for 40 seconds. The DNA fixing machine can provide the required “heat shock” by conduction. This can be used to replace limbs or reproduce cells that normally don't divide.
The fourth use of the DNA fixing machine is to increase the velocity of the blood by increasing the temperature of the blood (changing from laminar to turbulent flow) by conduction to fix problems at the blood vessels disorders (vascular diseases, clogged arteries, etc.)
Finally, the DNA fixing machine can be used to fix any problem where the cells inside the body are kept at a higher or lower temperature than the original cell temperature (sleeping viruses, etc,)
The DNA fixing machine can be used for humans and any kind of animal (animals have the same diseases as humans).
